(19) United States
(12) Patent Application Publication
(10) Pub. No.: US 2022/0193201 A1
(43) Pub. Date: Jun. 23, 2022

(54) GLP-1R AGONIST REDUCES RETINAL INFLAMMATION AND NEURON DEATH SECONDARY TO OCULAR HYPERTENSION

(71) Applicant: THE TRUSTEES OF THE UNIVERSITY OF PENNSYLVANIA, Philadelphia, PA (US)

(72) Inventors: Qi N. Cui, Yardley, PA (US); Joshua L. Dunaief, Wynnewood, PA (US); Jacob Sterling, Philadelphia, PA (US)

(73) Assignee: THE TRUSTEES OF THE UNIVERSITY OF PENNSYLVANIA, Philadelphia, PA (US)

(21) Appl. No.: 17/504,500

(22) Filed: Oct. 18, 2021

Related U.S. Application Data
(60) Provisional application No. 63/093,754, filed on Oct. 19, 2020.

(51) Int. Cl. A61K 38/26; A61K 9/00; A61P 27/16

(52) U.S. Cl. CPC A61K 38/26; A61P 27/16; A61K 9/0014

(57) ABSTRACT

Methods for reducing retinal inflammation and neuron death secondary to ocular hypertension, methods for reducing retinal inflammation and neuron death, reducing production of IL-1α, TNF-α, and C1q by CD11b+ CD11c+ and CD11b+ CD11c− cells during elevated intraocular pressure (IOP) and methods for decreasing transformation of astrocytes to an A1 neurotoxic phenotype (A1 astrocytes) and activation of the A1 astrocytes in a retina of a subject and decreasing production of complement component 3 (C3) by A1 astrocytes during elevated intraocular pressure in a subject in need thereof, comprising administering a glucagon-like peptide-1 receptor agonist.

### FIELD OF THE INVENTION

This invention relates to methods for reducing retinal inflammation and neuron death secondary to ocular hypertension in a subject in need thereof by administering a glucagon-like peptide-1 receptor agonist. This invention also relates to methods for reducing production of IL-1α, TNF-α, and C1q by CD11b+ CD11c+ and CD11b+ CD11c− cells during elevated intraocular pressure (IOP) in a subject, the method comprising administering to the subject a therapeutically effective amount of a glucagon-like peptide-1 receptor (GLP-1R) agonist. This invention further relates to methods for decreasing transformation of astrocytes to an A1 neurotoxic phenotype (A1 astrocytes) and activation of the A1 astrocytes in a retina of a subject and decreasing production of complement component 3 (C3) by A1 astrocytes during elevated intraocular pressure (eIOP), the method comprising administering to the subject a therapeutically effective amount of a glucagon-like peptide-1 receptor (GLP-1R) agonist.

### BACKGROUND OF THE INVENTION

Glaucoma is characterized by the death of retinal ganglion cells (RGCs) leading to permanent vision loss. It is the leading cause of irreversible blindness globally and is projected to affect approximately 112 million people worldwide by 2040. Elevated intraocular pressure (IOP) is strongly associated with glaucoma, and reduction of IOP is the only therapeutic mechanism available to slow disease progression. However, glaucoma can continue to progress even in patients who achieve normal IOP following medical and/or surgical treatments.

Reactive astrocytes are observed in multiple neurodegenerative diseases. In healthy neural tissue, astrocytes serve a wide variety of roles. They contribute to neurotransmitter recycling, neuronal metabolism, and formation of the blood-brain and blood-retina barriers. In the retina, astrocytes are found exclusively in the ganglion cell layer, comingled with RGCs. In response to both local and systemic stimuli, astrocytes can adopt reactive forms, A1 pro-inflammatory or A2 neuroprotective, both of which have been transcriptionally defined. A1 reactive astrocytes lose their phagocytic capacity as well as their ability to promote synapse formation and function. At the same time, A1 astrocytes gain pro-inflammatory and neurotoxic functions. In contrast, A2 astrocytes, observed in post-ischemic tissue, upregulate neurotrophic factors, promoting a neuroprotective environment. While A1 astrocytes have been implicated in multiple neurodegenerative diseases, their contribution to glaucoma has yet to elucidated and is being explored.

In light of the inadequacies of current drugs and therapies for glaucoma, there exists a critical need for improved compositions and therapeutically effective methods to prevent vision loss in patients suffering from glaucoma and to decrease or eliminate glaucoma progression in patients having normal IOP after standard therapies.

### SUMMARY OF THE INVENTION

In one aspect, this invention provides a method for reducing retinal inflammation and neuron death secondary to ocular hypertension in a subject in need thereof, the method comprising administering to the subject a therapeutically effective amount of a glucagon-like peptide-1 receptor (GLP-1R) agonist.

In another aspect, this invention provides a method for reducing production of IL-1α, TNF-α, and C1q by CD11b+ CD11c+ and CD11b+ CD11c− cells during elevated intraocular pressure (IOP) in a subject, the method comprising administering to the subject a therapeutically effective amount of a glucagon-like peptide-1 receptor (GLP-1R) agonist.

In yet another aspect, this invention provides a method for decreasing transformation of astrocytes to an A1 neurotoxic phenotype (A1 astrocytes) and activation of the A1 astrocytes in a retina of a subject and decreasing production of complement component 3 (C3) by A1 astrocytes during elevated intraocular pressure (eIOP), the method comprising administering to the subject a therapeutically effective amount of a glucagon-like peptide-1 receptor (GLP-1R) agonist.

In another aspect, this invention provides a method for reducing retinal ganglion cell (RGC) death secondary to elevated intraocular pressure (eIOP), decreasing or eliminating overexpression of IL-1α, TNF-α, and C1q in a retina and decreasing A1 astrocyte transformation in the retina of a subject having eIOP, the method comprising administering to the subject a therapeutically effective amount of an adenoviral vector comprising a triple knockout of genes encoding IL-1α, TNF-α, and C1q (I11a−/−; Tnf−/−; C1qa−/−).

Other features and advantages of this invention will become apparent from the following detailed description, examples, and figures. It should be understood, however, that the detailed description and specific examples while indicating certain embodiments of the invention are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIGS. 1A-1F show elevated intraocular pressure induces A1 astrocyte reactivity in the retina. C57BL6/J (WT) and I11a−/−; Tnf−/−; C1qa−/− knockout (TKO) mice were injected either with microbeads (“Bead”, left eye) into the anterior chamber (“AC”), to increase intraocular pressure (IOP), or with BSS (right eye). FIG. 1A shows IOP measurements across the duration of the study in both eyes of WT and TKO animals. Statistical difference for Bead WT vs BSS WT and Bead TKO vs BSS TKO is shown using stars. No statistical difference was detected between BSS WT and BSS TKO or Bead WT and Bead TKO. (n=20 eyes per condition per genotype). FIGS. 1B-1D show qPCR measurements of pan reactive, A1-specific, and A2 specific transcripts from ACSA2+ cells isolated from WT and TKO mice at 3 days (FIG. 1B), 14 days (FIG. 1C), and 42 days (FIG. 1D) post-injection (“p.i.”). (n=5 eyes per condition per genotype). FIG. 1E shows qPCR measurements of C3 mRNA levels in ACSA2+ cells 42 days post-injection. (n=5 eyes per condition per genotype). FIG. 1F shows ELISA measurements of C3 protein levels in ACSA2+ cells 42 days post-injection. (n=5 eyes per condition per genotype). All data presented as mean t SEM. Mann-Whitney U test, *p<0.05, **p<0.01, ***p<0.001, ****p<0.0001 vs. BSS WT. See also FIGS. 7A-7C.

FIGS. 2A-2B show TNF-α. IL-1α, and C1q-trigger retinal ganglion cell death secondary to eIOP. FIG. 2A shows C57BL6/J (WT), I11a−/−; Tnf−/−, C1qa−/− and I11a−/−; Tnf−/−; C1qa−/− knockout (TKO) mice were injected either with microbeads (left eye) into the anterior chamber (“AC”), to increase intraocular pressure (IOP), or with BSS (right eye). At 42 days post-injection mice were euthanized and retinal flatmounts were stained for the retinal ganglion cell (RGC) marker Brn3a (FIG. 2B). Cell count for each microbead-injected eye was normalized against the contralateral BSS-injected eye to determine percent survival of Brn3a+ cells. n=10 mice per genotype. All data presented as mean±SEM. One-way ANOVA with Tukey's multiple comparisons test, *p<0.05, **p<0.01, ***p<0.001. Representative images are shown on the right. Scale bar=50 μm.

FIGS. 3A-3F show early retinal inflammation is driven by CD11b+ CD11c+ cells and persists beyond re-normalization of IOP. C57BL6/J (WT) mice were injected either with microbeads (“Bead”, left eye) into the anterior chamber (“AC”), to increase intraocular pressure (IOP), or with BSS (right eye). FIGS. 3A-3C show ELISA measurements of IL-1α (FIG. 3A), TNF-α (FIG. 3B), or C1q (FIG. 3C) protein levels in whole neurosensory retina at 1, 2, 3, 5, 7, 14, 28, and 42 days post-injection. Statistical test compared BSS-injected eyes to microbead-injected eyes at the same time points. (n=5 eyes per time point per condition). FIGS. 3D-3F show CD11b+ CD11c− and CD11b+CD11c+ cells were isolated from neurosensory retina at 1, 2, 3, 5, 7, 14, 28, and 42 days post-injection. I11a (FIG. 3D), Tnf (FIG. 3E), and C1qa (FIG. 3F) mRNA levels were measured by qPCR in both cell populations. qPCR measurements in microbead-injected eyes were normalized to the contralateral BSS-injected eyes. Statistical tests compared microbead-injected CD11b+ CD11c+ cells and microbead-injected CD11b+ CD11c− cells at the same time points. (n=5 eyes per time point per condition). All data presented as mean t SEM. Mann-Whitney U test, *p<0.05, **p<0.01, ***p<0.001, ****p<0.0001. See also FIGS. 7A-7C, 8A-8C and 9A-9B.

FIGS. 4A-4H show GLP-1R agonist, NLY01, reduces acute production of IL-1α, TNF-α and C1q secondary to eIOP and A1 astrocyte activation. C57BL6/J (WT) mice were injected either with microbeads (“Bead”, left eye), to increase intraocular pressure (IOP), or with BSS (right eye). Following intraocular injections, mice were randomized to twice-weekly subcutaneous NLY01 (5 mg/kg per injection) or normal saline. Mice were euthanized 14 days post-injection. FIGS. 4A-4F show CD11b+ CD11c− and CD11b+ CD11c+ cells were isolated from neurosensory retina. qPCR was performed to measure I11a (FIGS. 4A, 4D), Tnf (FIGS. 4B, 4E) and C1qa (FIGS. 4C, 4F) mRNA levels in each population. (n=5 eyes per condition). FIG. 4G shows qPCR measurements of pan reactive, A1-specific and A2 specific transcripts from ACSA2+ cells 14 days post-injection. (n=5 eyes per condition). FIG. 4H shows qPCR measurement of C3 mRNA levels in ACSA2+ cells. (n=5 eyes per condition). All data presented as mean±SEM. Mann-Whitney U test, *p<0.05, **p<0.01, ***p<0.001, ****p<0.0001 relative to BSS NSS. See also FIGS. 7A-7C, 10 and 11A-11D.

FIGS. 5A-5H show NLY01 reduces CD11b+ CD11c+ and CD11b+ CD11c− IL-1α, TNF-α and C1q production due to prolonged eIOP and A1 astrocyte activation. C57BL6/J (WT) mice were injected either with microbeads (“Bead”, left eye), to increase intraocular pressure (IOP), or with BSS (right eye). Following injections, mice were randomized to twice weekly subcutaneous NLY01 (5 mg kg⁻¹ per injection) or normal saline. Mice were euthanized 42 days post-injection. FIGS. 5A-5F show CD11b+ CD11c− and CD11b+ CD11c+ cells were isolated from neurosensory retina. qPCR was performed to measure I11a (FIGS. 5A, 5D), Tnf (FIGS. 5B, 5E), and C1qa (FIGS. 5C, 5F) mRNA levels in each population. (n=5 eyes per condition). FIG. 5G shows qPCR measurements of pan reactive, A1-specific, and A2 specific transcripts from ACSA2+ cells 42 days post-injection. (n=5 eyes per condition). FIG. 5H shows qPCR measurement of C3 mRNA levels in ACSA2+ cells. (n=5 eyes per condition). All data presented as mean±SEM. Mann-Whitney U test, **p<0.01, ***p<0.001, ****p<0.0001 vs. BSS NSS. See also FIGS. 7A-7C, 10 and 11A-11D.

FIGS. 6A-6B show NLY01 reduces RGC death secondary to eIOP. C57BL6/J (WT) mice were injected either with microbeads (“Bead”, left eye), to increase intraocular pressure (IOP), or with BSS (right eye). Following injection, mice were randomized to twice weekly subcutaneous NLY01 (5 mg kg⁻¹ per injection) or normal saline. Mice were euthanized 42 days post-injection. FIG. 6A shows retinal flatmounts were stained for Brn3a. Cell count for each microbead-injected eye was normalized against the contralateral BSS-injected eye to determine percent survival of Bm3a+ cells. Representative images are shown on the right. Scale bar=50 μm. FIG. 6B shows retinal flatmounts were stained for Rbpms. Cell count for each microbead-injected eye was normalized against the contralateral BSS-injected eye to determine percent survival of Rbpms+ cells. Representative images are shown on the right. Scale bar=50 μm. n=13 NSS, n=15 NLY01. All data presented as mean±SEM. Mann-Whitney U test, ***p<0.001, ****p<0.0001 vs. BSS WT. See also FIG. 10.

FIGS. 7A-7C show a cell sorting paradigm related to FIGS. 1A-1F, 3A-3F, 4A-4H and 5A-5H. C57BL6/J (WT) mice were euthanized and neurosensory retina (“NSR”) were isolated and dissociated for cell sorting. FIG. 7A shows a cell isolation protocol using magnetic cell sorting. FIG. 7B shows qPCR measurements of cell-type specific markers in ACSA2+ cells. FIG. 7C shows qPCR measurements of cell-type specific markers in CD11b+ cells. N=8 eyes. All data presented as mean±SEM.

FIGS. 8A-8C show microbead-injection alone does not induce CD11b+ production of I11a, Tnf, or C1qa. Related to FIG. 3. C57BL6/J (WT) mice were injected either with microbeads (left eye), to increase intraocular pressure (IOP), or with BSS (right eye). IOP was monitored weekly. Bead eyes that did not have an IOP increase of 6 mmHg or greater within 2 weeks of injections were termed “non-OHT Bead” and excluded from cIOP studies. FIGS. 8A-5C show CD11b+ cells were isolated from neurosensory retina 42 days after injection and qPCR was performed to measure I11a (FIG. 8A), Tnf, (FIG. 8B) and C1qa (FIG. 8C) mRNA levels. N=5 eyes per condition. All data presented as mean±SEM.

FIGS. 9A-9B show CD11b+ CD11c− vs CD11b+ CD11c+ cell sorting paradigm. Related to FIG. 3. C57BL6/J (WT) mice were euthanized and neurosensory retina (“NSR”) were isolated and dissociated for cell sorting. FIG. 8A shows a cell isolation protocol using magnetic cell sorting. FIG. 8B shows qPCR measurement of Tmem119 mRNA levels in CD11b+ CD11c+ and CD11b+ CD11c− cells. N=5 eyes. All data presented as mean f SEM.

FIG. 10 shows NLY01 does not affect intraocular pressure. Related to FIGS. 4A-4H, 5A-5H and 6A-6B. C57BL6/J (WT) mice were injected either with microbeads (“Bead”, left eye), to increase intraocular pressure (IOP), or with BSS (right eye). Following intraocular injections, mice were randomized to twice weekly subcutaneous NLY01 at a dose of 5 mg kg¹ or normal saline solution (NSS). IOP were measured across the duration of the study. Statistical difference for Bead NSS vs BSS NSS and Bead NLY01 vs BSS NLY01 shown using stars. No statistical difference was detected between BSS NSS and BSS NLY01 or between Bead NSS and Bead NLY01. All data presented as mean t SEM. Mann-Whitney U test, ****p<0.0001. (n=25 eyes per condition per treatment)

FIGS. 11A-11D show NLY01 modulates NFκB signaling in CD11b+ CD11c− and CD11b+ CD11c+ cells. Related to FIGS. 4A-4H and 5A-5H. C57BL6/J (WT) mice were injected either with microbeads (“Bead”, left eye), to increase intraocular pressure (IOP), or with BSS (right eye). Following intraocular injections, mice were randomized to twice weekly subcutaneous NLY01 (5 mg kg⁻¹ per injection) or normal saline solution (NSS). 42 days post-injection mice were euthanized, neurosensory retina (“NSR”) was harvested and sorted as described in FIG. 9A to isolate CD11b+CD11c− and CD11b+ CD11c+ cells. FIG. 11A shows ELISA measurements of phospho-NFκB normalized to total NFκB in CD11b+ CD11c− cells. FIG. 11B shows ELISA measurements of phospho-NFκB normalized to total NFκB in CD11b+ CD11c+ cells. FIG. 11C shows qPCR measurement of NFκBIA rnRNA levels in CD11b+ CD11c− cells. FIG. 11D shows qPCR measurement of NFκBIA mRNA levels in CD11b+ CD11c+ cells. All data presented as mean±SEM. Ordinary one-way ANOVA, *p<0.05, **p<0.01, ***p<0.001, ****p<0.0001.

FIG. 12: Both eyes of C57BL6/J mice were injected with either microbeads (“Beads”) to increase intraocular pressure (IOP) or with balanced salt solution (“BSS”) to serve as normal pressure controls. One eye of each animal was treated with liraglutide (twice daily to the corneal surface) while the fellow eye was treated with BSS. After 6 weeks, retinal flatmounts were stained for the retinal ganglion cell (RGC) marker RBPMS. Results show that topical treatment with the GLP-1R agonist liraglutide reduced RGC loss in a hypertensive mouse glaucoma model. Mean±SEM, * p<0.01.

### DETAILED DESCRIPTION OF THE INVENTION

The present invention may be understood more readily by reference to the following detailed description which forms a part of this disclosure. It is to be understood that this invention is not limited to the specific methods, products, conditions or parameters described and/or shown herein, and that the terminology used herein is for the purpose of describing particular embodiments by way of example only and is not intended to be limiting of the claimed invention.

Recent studies have implicated pro-inflammatory microglia, macrophages, and A1 astrocytes in the pathogenesis of neurodegenerative diseases. The role of pro-inflammatory, neurotoxic A1 astrocytes in glaucoma is beginning to be investigated.

In the brain and the retina, neurotoxic A1 astrocytes are induced by microglial release of pro-inflammatory cytokines IL-1α, TNF-α, and C1q. Strong links exist between these three cytokines and glaucoma. ILIA and TNF polymorphisms are associated with primary open angle glaucoma. TNF-α protein levels are elevated in the vitreous, retina, and optic nerves of glaucomatous eyes. In the DBA/2J mouse model of hypertensive glaucoma, C1qa mRNA levels are associated with disease progression and C1q inhibition is sufficient to prevent early RGC synapse loss and RGC death. C1q upregulation has been demonstrated in glaucomatous human eyes. Although IL-1α, TNF-α and C1q have been independently implicated in glaucoma, it is not known whether they act in concert to induce A1 astrocyte reactivity in glaucomatous retinas.

Glucagon-like peptide 1 is an incretin hormone that regulates blood glucose, weight, and satiety through its action at the glucagon-like peptide 1 receptor (GLP-1R) in both the systemic circulation and the central nervous system. NLY01 is a long-acting GLP-1R agonist with an extended half-life and favorable blood-brain barrier penetration. In mouse models of Parkinson's disease (PD), A1 astrocytes contribute to dopaminergic cell death and poor motor phenotypes. NLY01 has been shown to reduce microglial production of C1q, TNF-α, and IL-1α, thereby blocking A1 astrocyte transformation, reducing dopaminergic cell death, and improving motor symptoms in mouse models of PD.

Using a microbead-induced ocular hypertension mouse glaucoma model, it is shown herein that microglia and infiltrating macrophages upregulate C1q, TNF-α, and IL-1a. These three cytokines are necessary for A1 transformation within the retina. Cytokine upregulation and A1 transformation persist despite normalization of IOP six weeks post-injection. Genetic deletion of these cytokines prevents A1 astrocyte formation and RGC loss. Finally, NLY01 therapy reduced CD11b+ CD11c− and CD11b+ CD11c+ production of C1q, TNF-α, and IL-1α, A1 astrocyte transformation, and RGC loss in the present model. Together, these data demonstrate that GLP-1R activation is capable of reducing ocular inflammation driven by both CD11b+ CD11c− and CD11b+ CD11c+ cell populations, and thus preventing A1 astrocyte activation and rescuing RGCs from hypertensive glaucoma. NLY01 has potential clinical use in the treatment of glaucoma and possibly other retinal diseases characterized by reactive astrogliosis.

The present invention is directed to the treatment of glaucoma by administration of a glucagon-like peptide-1 receptor (GLP-1R) agonist.to subjects in need thereof. While not intending to be bound by any particular mechanism of operation, it is believed that the GLP-1R agonist administered according to the present invention reduces retinal inflammation and neuron death secondary to ocular hypertension that occurs in glaucoma. In particular, administration of a GLP-1R agonist reduces production of IL-1α, TNF-α, and C1q by CD11b+ CD11c+ and CD11b+ CD1l c− cells during elevated intraocular pressure (eIOP).

The present invention also is directed to the treatment of glaucoma by administration of an adenoviral vector comprising a triple knockout of genes encoding IL-1α, TNF-α, and C1q (I11a−/−; Tnf−/−; C1qa−/−). Without intending to be bound by any particular mechanism of operation, it is believed that the adenoviral vector comprising a triple knockout of genes encoding IL-1α, TNF-α, and C1q administered according to the present invention reduces retinal ganglion cell (RGC) death secondary to the elevated IOP by decreasing or eliminating overexpression of IL-1α, TNF-α, and C1q in the retina, and/or by decreasing A1 astrocyte transformation in the retina of subjects having glaucoma with an eIOP.

Unless otherwise defined herein, scientific, and technical terms used in connection with this application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In this disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality,” as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

In one aspect, this invention provides a method for reducing retinal inflammation and neuron death secondary to ocular hypertension in a subject in need thereof, the method comprising administering to the subject a therapeutically effective amount of a glucagon-like peptide-1 receptor (GLP-1R) agonist. In an embodiment, the GLP-1R agonist is a pegylated exendin-4 (exenatide), NLY01. In some embodiments, the GLP-1R agonist is exenatide, lixisenatide, liraglutide, albiglutide, dulaglutide, or semaglutide. In some embodiments, the GLP-1R agonist is pegylated. In certain embodiments, the GLP-1R agonist is fused to human serum albumin, i.e., albuminated. In an embodiment, the GLP-1R agonist is pegylated liraglutide (QPG-1029). In another embodiment, the GLP-1R agonist is a modified human GLP-1, wherein the modified human GLP-1 is albuminated, wherein the albuminated modified human GLP-1 is albiglutide. In an embodiment, the GLP-1R agonist is GLP-1(7-37) covalently linked to an Fc fragment of human IgG4, wherein the GLP-1(7-37) covalently linked to the Fc fragment of human IgG4 is dulaglutide. In another embodiment, the method further comprises reducing the ocular hypertension from an intraocular pressure (IOP) of greater than 21 mm Hg by 20% to 30%. In an embodiment, the intraocular pressure is reduced by topically administering an ocular hypotensive medication to an eye of the subject. In some embodiments, the ocular hypotensive medication is a prostaglandin analog, a beta-adrenergic blocking agent, an alpha adrenergic agonist, a carbonic anhydrase inhibitor, a Rho kinase inhibitor or combinations thereof. In an embodiment, the prostaglandin analog is selected from the group consisting of latanoprost, travoprost, tafluprost, bimatoprost and latanoprostene bunod. In certain embodiments, the beta-adrenergic blocking agent is selected from the group consisting of timolol, levobunolol, betaxolol, carteolol, metipranolol, and levobetaxolol. In an embodiment, the alpha adrenergic agonist is brimonidine. or apraclonidine. In some embodiments, the carbonic anhydrase inhibitor is selected from the group consisting of dorzolamide, brinzolamide, acetazolamide, and methazolamide. In an embodiment, the Rho kinase inhibitor is Ripasudil (K-115) or Netarsudil (AR-13503). In some embodiments, the combinations of ocular hypotensive medication comprise a beta blocker and a carbonic anhydrase inhibitor; an alpha adrenergic agonist and a beta blocker; or a carbonic anhydrase inhibitor and an alpha adrenergic agonist. In an embodiment, the intraocular pressure (IOP) is reduced by performing glaucoma surgery Laser peripheral iridotomy (LPI), drainage implant surgery, electrocautery or a non-penetrating glaucoma surgery, wherein the non-penetrating glaucoma surgery comprises deep sclerectomy or viscocanalostomy. In certain embodiments, the GLP-1R agonist is administered to the subject following administration of the ocular hypotensive medication to the eye of the subject, wherein the ocular hypertension is reduced to a normal intraocular pressure of less than 21 mm Hg. In an embodiment, the GLP-1R agonist is administered to the subject following the glaucoma surgery on the eye of the subject, wherein the ocular hypertension is reduced to a normal intraocular pressure of less than 21 mm Hg.

In another aspect, this invention provides a method for reducing production of IL-1α, TNF-α, and C1q by CD11b+ CD11c+ and CD11b+ CD11c− cells during elevated intraocular pressure (IOP) in a subject, the method comprising administering to the subject a therapeutically effective amount of a glucagon-like peptide-1 receptor (GLP-1R) agonist. In an embodiment, the GLP-1R agonist is a pegylated exendin-4 (exenatide), NLY01. In another embodiment, the GLP-1R agonist is exenatide, lixisenatide, liraglutide, albiglutide, dulaglutide, or semaglutide. In some embodiments, the GLP-1R agonist is pegylated. In certain embodiments, the GLP-1R agonist is fused to human serum albumin, i.e., albuminated. In an embodiment, the GLP-1R agonist is pegylated liraglutide (QPG-1029). In another embodiment, the GLP-1R agonist is a modified human GLP-1, wherein the modified human GLP-1 is albuminated, wherein the albuminated modified human GLP-1 is albiglutide. In an embodiment, the GLP-1R agonist is GLP-1(7-37) covalently linked to an Fc fragment of human IgG4, wherein the GLP-1(7-37) covalently linked to the Fc fragment of human IgG4 is dulaglutide. In an embodiment, the intraocular pressure (IOP) is reduced by performing glaucoma surgery Laser peripheral iridotomy (LPI), drainage implant surgery, electrocautery or a non-penetrating glaucoma surgery, wherein the non-penetrating glaucoma surgery comprises deep sclerectomy or viscocanalostomy. In certain embodiments, the GLP-1R agonist is administered to the subject following administration of the ocular hypotensive medication to the eye of the subject, wherein the ocular hypertension is reduced to a normal intraocular pressure of less than 21 mm Hg. In an embodiment, the GLP-1R agonist is administered to the subject following the glaucoma surgery on the eye of the subject, wherein the ocular hypertension is reduced to a normal intraocular pressure of less than 21 mm Hg.

In another aspect, this invention provides a method for reducing production of IL-1α, TNF-α, and C1q by CD11b+ CD11c+ and CD11b+ CD11c− cells during elevated intraocular pressure (IOP) in a subject, the method comprising administering to the subject a therapeutically effective amount of a glucagon-like peptide-1 receptor (GLP-1R) agonist. In an embodiment, the GLP-1R agonist is a pegylated exendin-4 (exenatide), NLY01. In another embodiment, the GLP-1R agonist is exenatide, lixisenatide, liraglutide, albiglutide, dulaglutide, or semaglutide. In some embodiments, the GLP-1R agonist is pegylated. In certain embodiments, the GLP-1R agonist is fused to human serum albumin, i.e., albuminated. In an embodiment, the GLP-1R agonist is pegylated liraglutide (QPG-1029). In another embodiment, the GLP-1R agonist is a modified human GLP-1, wherein the modified human GLP-1 is albuminated, wherein the albuminated modified human GLP-1 is albiglutide. In an embodiment, the GLP-1R agonist is GLP-1(7-37) covalently linked to an Fc fragment of human IgG4, wherein the GLP-1(7-37) covalently linked to the Fc fragment of human IgG4 is dulaglutide. In some embodiments, the method further comprises reducing the ocular hypertension from an intraocular pressure (IOP) of greater than 21 mm Hg by 20% to 30%. In an embodiment, the intraocular pressure is reduced by topically administering an ocular hypotensive medication to an eye of the subject. In another embodiment, the ocular hypotensive medication is a prostaglandin analog, a beta-adrenergic blocking agent, an alpha adrenergic agonist, a carbonic anhydrase inhibitor, a Rho kinase inhibitor or combinations thereof. In an embodiment, the prostaglandin analog is selected from the group consisting of latanoprost, travoprost, tafluprost, bimatoprost and latanoprostene bunod. In some embodiments, the beta-adrenergic blocking agent is selected from the group consisting of timolol, levobunolol, betaxolol, carteolol, metipranolol, and levobetaxolol. In an embodiment, the alpha adrenergic agonist is brimonidine. or apraclonidine. In various embodiments, the carbonic anhydrase inhibitor is selected from the group consisting of dorzolamide, brinzolamide, acetazolamide, and methazolamide. In an embodiment, the Rho kinase inhibitor is Ripasudil (K-115) or Netarsudil (AR-13503). In some embodiments, the combinations of ocular hypotensive medication comprise a beta blocker and a carbonic anhydrase inhibitor, an alpha adrenergic agonist and a beta blocker; or a carbonic anhydrase inhibitor and an alpha adrenergic agonist. In an embodiment, the elevated intraocular pressure (IOP) is reduced by performing glaucoma surgery on an eye of the subject. In certain embodiments, the glaucoma surgery is Selective Laser Trabeculoplasty (SLT), argon laser trabeculoplasty (ALT), trabeculectomy, cyclophotocoagulation, Laser peripheral iridotomy (LPI), drainage implant surgery, electrocautery or a non-penetrating glaucoma surgery, wherein the non-penetrating glaucoma surgery comprises deep sclerectomy or viscocanalostomy. In some embodiments, the GLP-1R agonist is administered to the subject following administration of the ocular hypotensive medication to the eye of the subject, wherein the ocular hypertension is reduced to a normal intraocular pressure of less than 21 mm Hg. In certain embodiments, the GLP-1R agonist is administered to the subject following the glaucoma surgery on the eye of the subject, wherein the ocular hypertension is reduced to a normal intraocular pressure of less than 21 mm Hg.

In yet another aspect, this invention provides a method for decreasing transformation of astrocytes to an A1 neurotoxic phenotype (A1 astrocytes) and activation of the A1 astrocytes in a retina of a subject and decreasing production of complement component 3 (C3) by A1 astrocytes during elevated intraocular pressure (eIOP), the method comprising administering to the subject a therapeutically effective amount of a glucagon-like peptide-1 receptor (GLP-1R) agonist. In a particular embodiment, the GLP-1R agonist is a pegylated exendin-4 (exenatide), NLY01. In some embodiments, the GLP-1R agonist is exenatide, lixisenatide, liraglutide, albiglutide, dulaglutide, or semaglutide. In some embodiments, the GLP-1R agonist is pegylated. In certain embodiments, the GLP-1R agonist is fused to human serum albumin, i.e., albuminated. In an embodiment, the GLP-1R agonist is pegylated liraglutide (QPG-1029). In another embodiment, the GLP-1R agonist is a modified human GLP-1, wherein the modified human GLP-1 is albuminated, wherein the albuminated modified human GLP-1 is albiglutide. In an embodiment, the GLP-1R agonist is GLP-1(7-37) covalently linked to an Fc fragment of human IgG4, wherein the GLP-1(7-37) covalently linked to the Fc fragment of human IgG4 is dulaglutide. In some embodiments, the method further comprises reducing the ocular hypertension from an intraocular pressure (IOP) of greater than 21 mm Hg by 20% to 30%. In an embodiment, the intraocular pressure is reduced by topically administering an ocular hypotensive medication to an eye of the subject. In another embodiment, the ocular hypotensive medication is a prostaglandin analog, a beta-adrenergic blocking agent, an alpha adrenergic agonist, a carbonic anhydrase inhibitor, a Rho kinase inhibitor or combinations thereof. In various embodiments, the prostaglandin analog is selected from the group consisting of latanoprost, travoprost, tafluprost, bimatoprost and latanoprostene bunod. In some embodiments, the beta-adrenergic blocking agent is selected from the group consisting of timolol, levobunolol, betaxolol, carteolol, metipranolol, and levobetaxolol. In an embodiment, the alpha adrenergic agonist is brimonidine. or apraclonidine. In some embodiments, the carbonic anhydrase inhibitor is selected from the group consisting of dorzolamide, brinzolamide, acetazolamide, and methazolamide. In an embodiment, the Rho kinase inhibitor is Ripasudil (K-115) or Netarsudil (AR-13503). In certain embodiments, the combinations of ocular hypotensive medication comprise a beta blocker and a carbonic anhydrase inhibitor, an alpha adrenergic agonist and a beta blocker, or a carbonic anhydrase inhibitor and an alpha adrenergic agonist. In some embodiments, the intraocular pressure (IOP) is reduced by performing glaucoma surgery on an eye of the subject. In certain embodiments, the glaucoma surgery is Selective Laser Trabeculoplasty (SLT), argon laser trabeculoplasty (ALT), trabeculectomy, cyclophotocoagulation, Laser peripheral iridotomy (LPI), drainage implant surgery, electrocautery or a non-penetrating glaucoma surgery, wherein the non-penetrating glaucoma surgery comprises deep sclerectomy or viscocanalostomy. In an embodiment, the GLP-1R agonist is administered to the subject following administration of the ocular hypotensive medication to the eye of the subject, wherein the ocular hypertension is reduced to a normal intraocular pressure of less than 21 mm Hg. In another embodiment, the GLP-1R agonist is administered to the subject following the glaucoma surgery on the eye of the subject, wherein the ocular hypertension is reduced to a normal intraocular pressure of less than 21 mm Hg.

In another aspect, this invention provides a method for reducing retinal ganglion cell (RGC) death secondary to elevated intraocular pressure (eIOP), decreasing or eliminating overexpression of IL-1α, TNF-α, and C1q in a retina, and decreasing A1 astrocyte transformation in the retina of a subject having cIOP, the method comprising administering to the subject a therapeutically effective amount of an adenoviral vector comprising a triple knockout of genes encoding IL-1α, TNF-α, and C1q (I11a−/−; Tnf−/−; C1qa−/−). In an embodiment, the adenoviral vector comprising the I11a−/−; Tnf−/−; C1qa−/− is constructed by CRISPR-cas9 based genome editing to delete I11a; Tnf; and C1qa.

### Pharmaceutical Compositions

Described herein are pharmaceutical compositions comprising compounds or of the invention and one or more pharmaceutically acceptable carriers and methods of administering them. “Pharmaceutically acceptable carriers” include any excipient which is nontoxic to the cell or mammal being exposed thereto at the dosages and concentrations employed. The pharmaceutical composition may include one or more therapeutic agents. In an embodiment, the therapeutic agent comprises a glucagon-like peptide-1 receptor (GLP-1R) agonist. In a particular embodiment, the therapeutic agent comprises GLP-1R agonist pegylated exendin-4 (exenatide), NLY01.

Thus, as used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In an embodiment, pharmaceutical compositions containing the therapeutic agent or agents described herein, can be, in one embodiment, administered to a subject by any method known to a person skilled in the art, such as, without limitation, orally, parenterally, transnasally, transmucosally, subcutaneously, transdermally, intramuscularly, intravenously, intraarterially, intra-dermally, intra-peritoneally, intra-ventricularly, intra-cranially, or intra-vaginally.

Carriers may be any of those conventionally used, as described above, and are limited only by chemical-physical considerations, such as solubility and lack of reactivity with the compound of the invention, and by the route of administration. The choice of carrier will be determined by the particular method used to administer the pharmaceutical composition. Some examples of suitable carriers include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. The formulations can additionally include lubricating agents such as talc, magnesium stearate, and mineral oil; wetting agents, surfactants, emulsifying and suspending agents; preserving agents such as methyl- and propylhydroxybenzoates; sweetening agents; flavoring agents, colorants, buffering agents (e.g., acetates, citrates or phosphates), disintegrating agents, moistening agents, antibacterial agents, antioxidants (e.g., ascorbic acid or sodium bisulfite), chelating agents (e.g., ethylenediaminetetraacetic acid), and agents for the adjustment of tonicity such as sodium chloride. Other pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. In one embodiment, water, preferably bacteriostatic water, is the carrier when the pharmaceutical composition is administered intravenously or intratumorally. Saline solutions and aqueous dextrose and glycerol solutions can also be employed as liquid carriers, particularly for injectable solutions.

Pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include, without limitation, physiological saline, bacteriostatic water, Cremophor EL.TM. (BASF, Parsippany, N.J.) or phosphate buffered saline (PBS). The composition should be sterile and should be fluid to the extent that easy syringeability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as appropriate, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

The compositions and formulations as described herein may be administered alone or with other biologically active agents. Administration can be systemic or local, e.g. through portal vein delivery to the liver. In addition, it may be advantageous to administer the composition into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an acrosolizing agent. It may also be desirable to administer the Therapeutic locally to the area in need of treatment; this may be achieved by, for example, and not by way of limitation, local infusion during surgery, topical application, by injection, by means of a catheter, by means of a suppository, or by means of an implant.

Moreover, “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable” also includes those carriers approved by a regulatory agency of the Federal or a state government or listed in the U.S. Pharmacopeia or other generally recognized pharmacopeia for use in animals and, more particularly, in humans.

### Effective Doses

Effective doses of the pharmaceutical compositions of the present invention, for treatment of conditions or diseases vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy. The pharmaceutical compositions of the invention thus may include a “therapeutically effective amount.” A “therapeutically effective amount” refers to an amount effective, at dosages and for periods of time necessary, to achieve the desired therapeutic result. A therapeutically effective amount of a molecule or therapeutic agent may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

Furthermore, a skilled artisan would appreciate that the term “therapeutically effective amount” may encompass total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of a compound of the invention that will be effective in the treatment of a particular disorder or condition, including retinal inflammation and neuron death secondary to ocular hypertension, also will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. In one embodiment, the dosage of the glucagon-like peptide-1 receptor (GLP-1R) agonist, the ocular hypotensive medication, including a prostaglandin analog, a beta-adrenergic blocking agent, an alpha adrenergic agonist, a carbonic anhydrase inhibitor, a Rho kinase inhibitor or combinations thereof, will be within the range of about 0.01-about 1000 mg/kg of body weight. In another embodiment, the dosage will be within the range of about 0.1 mg/kg to about 100 mg/kg. In another embodiment, the dosage will be within the range of about 1 mg/kg to about 10 mg/kg. In an embodiment, the dosage is about 10 mg/kg. In another embodiment, the dosage is 10 mg/kg.

The compound or composition or therapeutic agent of the invention, including the GLP-1R) agonist, may be administered only once, or it may be administered multiple times. For multiple dosages, the composition may be, for example, administered three times a day, twice a day, once a day, once every two days, twice a week, weekly, once every two weeks, or monthly.

In an embodiment, the dosage is administered twice daily. In an embodiment, the dosage is administered for four weeks. In an embodiment, the dosage is 10 mg/kg and is administered twice daily for four weeks. The dosage may be administered for 1 week, ten days, two weeks, three weeks, four weeks, six weeks, eight weeks or more, as needed to achieve the desired therapeutic effect. Moreover, effective doses may be extrapolated from dose-response curves derived from in vitro or animal model test bioassays or systems.

All scientific publications cited herein are hereby incorporated by reference in their entireties.

The following examples are presented in order to illustrate certain embodiments of the invention more fully. The examples should in no way be construed, however, as limiting the broad scope of the invention.

### EXAMPLES

### Methods and Materials

### Experimental Model

### Mice

All mice were adult (>3 months old), age-, strain- and sex-matched. C57BL6/J (WT) mice were obtained from Jackson labs (Stock Number 000664). I11a−/−; Tnf−/−, C1qa−/−, and I11a−/−; Tnf−/−; C1qa−/− (TKO) animals were generously donated by Ben Barres (Stanford University). All animals were fed ad libitum and maintained on a 12 h/12 h light/dark cycle in a University of Pennsylvania vivarium. All procedures were approved by the Institutional Animal Care and Use Committee of the University of Pennsylvania and complied with the ARVO Statement for the Use of Animals in Ophthalmic and Vision Research. NLY01 was obtained through a material transfer agreement with Neuraly (Baltimore, Md.). A cohort of mice was treated with either twice weekly subcutaneous injections of NLY01 (5 mg kg⁻¹ per injection) or with an equivalent amount of normal saline solution (L21819; Fisher Scientific).

### Method Details

### Anterior Chamber Injection and Intraocular Pressure (IOP) Measurement

The microbead occlusion model was used to induce elevated intraocular pressure as described previously. Briefly, mice were anesthetized with intraperitoneal injections of ketamine (80 mg/kg, Par Pharmaceutical), xylazine (10 mg/kg, Lloyd), and acepromazine (2 mg/kg, Boehringer Ingelheim Vetmedica). Pupils were dilated with topical 1% tropicamide and 2.5% phenylephrine (Akorn). Proparacaine anesthetic eye drop at a concentration of 0.5% (Sandoz) were applied immediately prior to injection. Injection micropipettes were pulled from glass capillaries to a final diameter of ˜100 μm and connected to a microsyringe pump. Using a micromanipulator for positioning, one eye of the mouse was injected with 1.5 μL of sterile 4.5 μm-diameter magnetic microbeads (1.6×10⁶ beads/μL of balanced salt solution; Thermo Fisher Scientific) at a location <1 mm central to the limbus, while the other eye was injected with an equivalent volume of balanced salt solution (Alcon Laboratories). A hand-held magnet was used to target the beads into the drainage angle. After injection, 0.5% moxifloxacin antibiotic drops (Sandoz) were applied to the eye. IOP was measured between 8 and 11 a.m. using the Icare TONOLAB tonometer (Icare TONOVET). An average of three measurements/eye was used.

### Retinal Cell Sorting

ACSA2+, CD11b+ CD11c−, and CD11b+ CD11c+ cells were isolated from adult murine retinas using the Miltenyi Adult Brain Dissociation Kit (Miltenyi Biotec, 130-107-677) and MACS magnetic cell separation system. Briefly, mice were anesthetized, euthanized, and whole neurosensory retinas were harvested. Retinal tissue was dissociated in manufacturer provided enzyme mixtures using the gentleMACS dissociator pre-set program: 37C_ABDK_02. Retinal cell suspensions were passed through a 70 μm filter (130-098-462, Miltenyi Biotec) and resuspended in debris removal solution. Following debris removal, retinal cells were resuspended and incubated in red blood cell removal solution for 10 mins at 4° C. The following isolation steps were performed: (1) positive selection for ACSA2 (astrocyte and Muller cell enriched, 130-097-678, Miltenyi Biotec), (2) the remaining negative selection pool was then subjected to positive selection for CD11b (microglia and macrophage enriched, 130-093-636 Miltenyi Biotec), and (3) the CD11b+ cells were then selected against CD11c (130-125-835, Miltenyi Biotec). This resulted in three cell populations: (1) ACSA2+, (2) ACSA2− CD11b+ CD11c−, and (3) ACSA2− CD11b+ CD11c+. Positive and negative selection steps used MACS magnetic separation LS columns (130-042-401, Miltenyi Biotech) according to the manufacturer's protocol. Cells were subsequently used for RNA isolation using the RNeasy Mini Kit (Qiagen), according to the manufacturer's protocol.

Quantitative PCR (qPCR)

RNA isolation was performed according to the manufacturer's protocol (RNeasy kit; Qiagen). cDNA was synthesized with reverse transcription agents (TaqMan Reverse Transcription Reagents, Applied Biosystems) according to the manufacturer's protocol. Realtime qPCR (TaqMan; ABI) was performed on a sequence detection system (Prism Model 7500; ABI) using the ΔΔCT method, which provided normalized expression values (normalized against Gapdh). All reactions were performed in technical triplicates (three qPCR replicates per qPCR probe).

### Enzyme-Linked Immunosorbent Assays

ELISA kits were used to measure protein levels of IL-1α (BMS627, Thermofisher Scientific), TNF-α (BMS607-3, Thermofisher Scientific), C1q (LS-F55223-1, LifeSpan BioSciences), C3 (ab157711, Abcam), and phospho-NFκB/total NFκB (Thermofisher Scientific, 50-246-259) according to the manufacturer's protocol. Briefly, mouse retinas were collected, in select cases subjected to cell sorting, and subsequently homogenized in phosphate-buffered saline containing the protease inhibitor phenylmethylsulfonylfluoride (100 lM; EMD, Gibbstown, N.J., USA). Assays were performed according to the manufacturers' protocol. Protein levels were determined by comparing the absorbance produced by the samples with that of a calibration curve. All measurements were performed in technical triplicate.

### Preparation of Retinal Flatmounts, Immunofluorescence & Cell Counting

RGC quantification and immunolabeling of flat-mounted retinas were performed as described by Cui et al., 2020, Exp Eye Res 193, 107961, which is incorporated herein by reference in its entirety. Briefly, eyes were enucleated and fixed in 4% paraformaldehyde. Retinas were isolated, mounted on glass slides and serially washed with 0.5% Triton X-100 in phosphate-buffered saline (PBS). Flat-mounted retinas were incubated overnight at 4° C. with antibodies against RNA-binding protein with multiple splicing (RBPMS; EMD Millipore) diluted 1:500 in blocking buffer (2% bovine serum albumin, 2% Triton X-100 in PBS) and brain-specific homeobox/POU domain protein-3a (Brn3a; Synaptic Systems) diluted 1:1000 in blocking buffer. The following day, retinas were washed and incubated with Alexa Fluor 488 donkey anti-rabbit IgG (Invitrogen; 1:1000 in blocking buffer) and Cy3 goat anti-guinea pig IgG (Abeam; 1:500 in blocking buffer) secondary antibodies for 3 hours at room temperature. After serial washes, flat-mounts were cover-slipped with Vectashield mounting medium containing DAPI (Vector Laboratories). For each flat-mount, 12 standardized photomicrographs were taken at 1/6, 3/6, and 5/6 distance from the center of the retina at 40-magnification by a masked operator. A masked counter quantified the number of Rbpms- and Brn3a-positive cells in each 40× field (0.069 mm²) using Nikon Elements analysis software version 4.1 (Nikon Instruments). The average number of cells in the 12 standardized photomicrographs from the microbead-injected eye was normalized to the average number of cells in the 12 standardized photomicrographs from the BSS-injected eye to calculate percent survival in the microbead injected eye with eIOP.

### Quantification and Statistical Analysis

All statistical analyses were done using GraphPad Prism 8.0 software. Data was analyzed either by one-way ANOVA followed by Tukey's multiple comparisons test for comparing between three or more samples, or Mann-Whitney U test for comparing between two samples with 95% confidence without assuming a gaussian distribution. Power calculations were performed using G® Power Software V 3.1.9.7, as described by Faul et al., 2007, Behav Res Methods 39, 175-191, which is incorporated herein by reference in its entirety. Group sizes were calculated to provide at least 80% power with the following parameters: probability of type I error (0.05), effect size (0.25).

### Example 1

### Elevated Intraocular Pressure Induces A1 Astrocyte Reactivity in the Retina

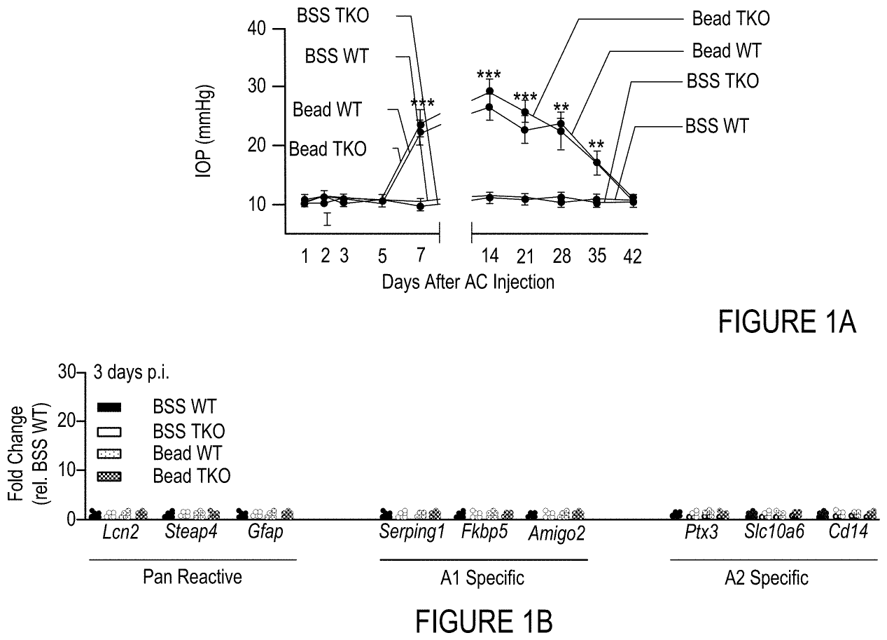

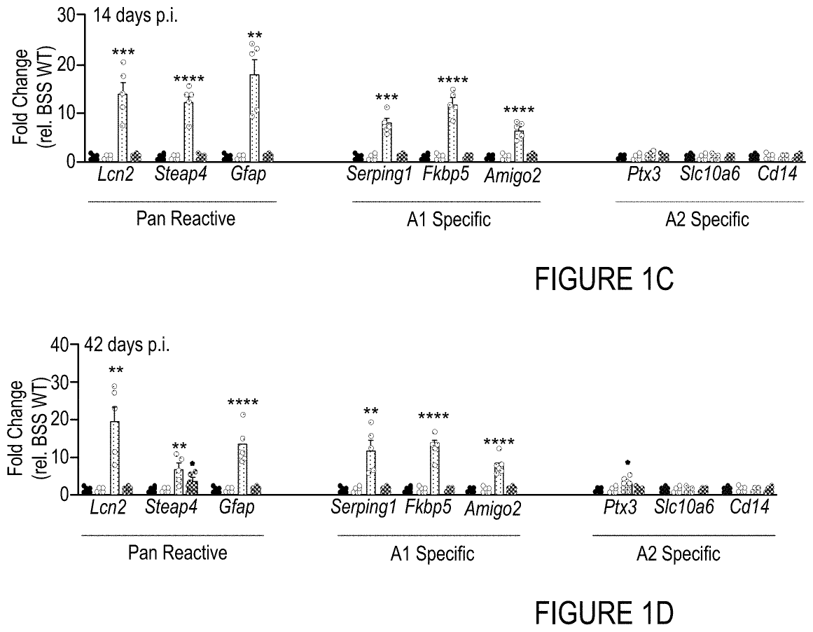

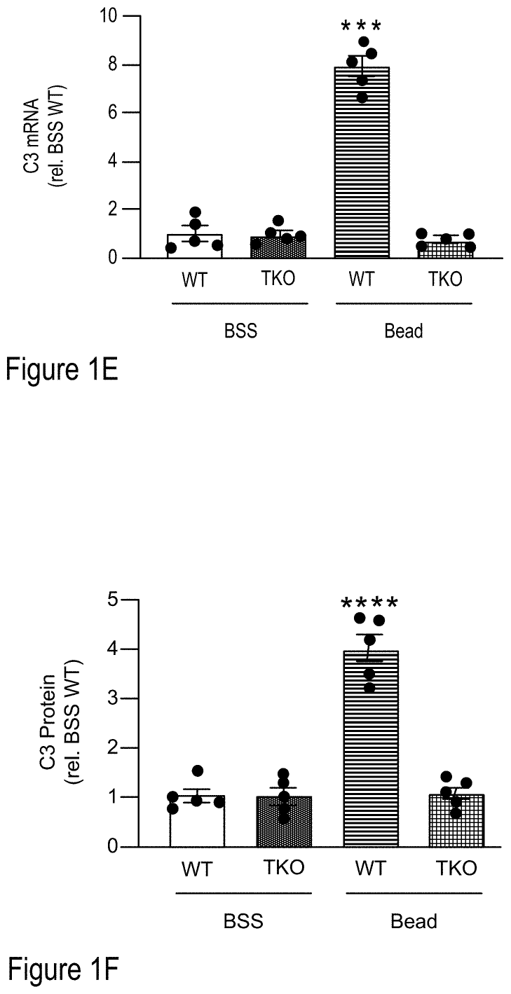

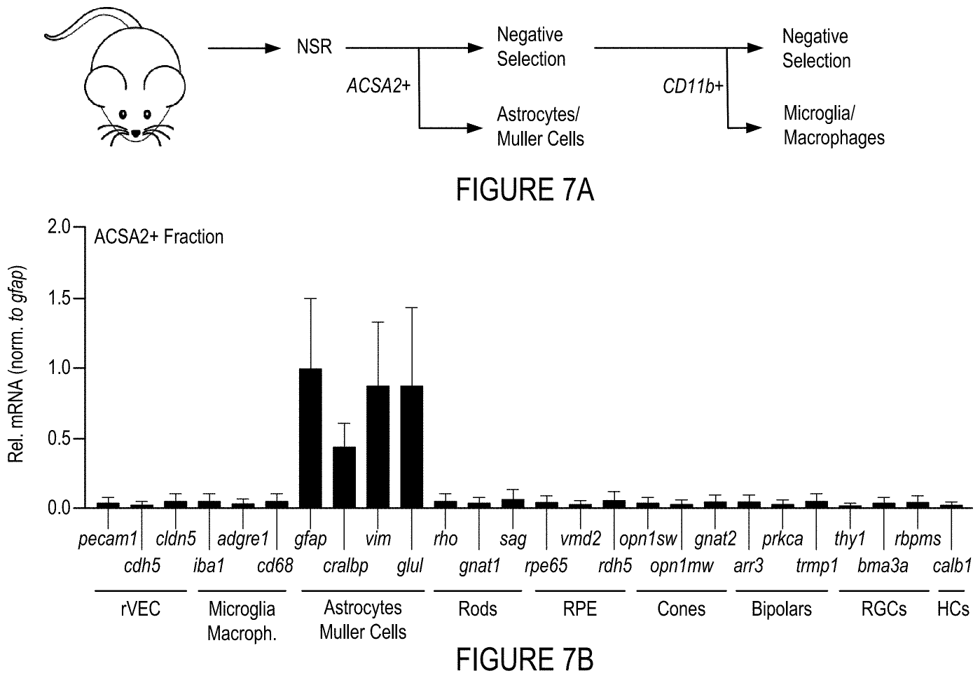

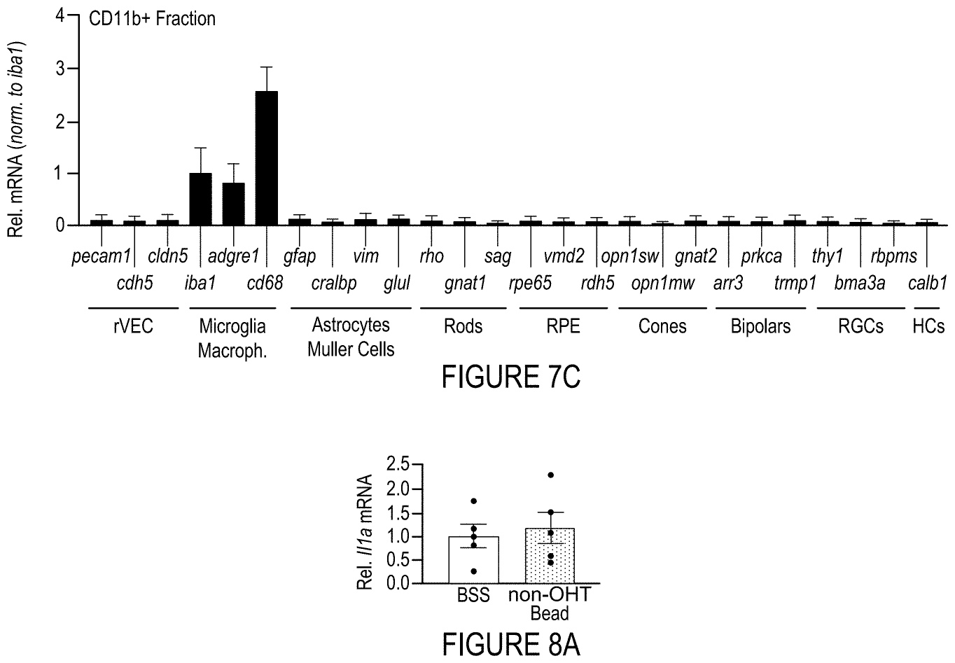

Magnetic microbeads (left eye) or balanced salt solution (BSS, right eye) were injected into the anterior chamber (AC) of wild-type (WT) and I11a−/−; Tnf−/−; C1qa−/− triple knockout (TKO) mice. Intraocular pressure (IOP) was recorded at 1, 2, 3, 5, and 7 days post-injection, and weekly thereafter. Microbead-injected eyes (Bead) in both WT and TKO animals had elevated intraocular pressures (eIOPs) beginning at 7 days post-injection compared to BSS-injected eyes (BSS) (FIG. 1A). IOPs peaked at 14 days post-injection and remained elevated throughout the 35 days post-injection. There was no difference in IOP between Bead and BSS eyes by the 42nd day post-injection (FIG. 1A). Neurosensory retinas were isolated for cell sorting 3, 14, and 42 days post-injection (p.i.) (FIG. 7A). Cell-type enrichment for all fractions used were validated by qPCR (FIGS. 7B-7C). Astrocytes and Müller cells were isolated using ASCA2+ selection, as reported previously. By the 14th day post-injection, ACSA2+ cells exhibited increased levels of pan reactive and A1 astrocyte specific markers (FIGS. 1B-1D), suggesting that A1 astrocyte transformation occurs early in the disease process. A1 reactivity persisted in microbead-injected WT animals 42 days post-injection, despite normalization of IOP (FIG. 1D). TKO animals failed to form A1 astrocytes despite eIOP (FIG. 1C-1D). This is consistent with previous findings. Markers of A2 reactivity were consistently unchanged in WT microbead-injected eyes compared to WT BSS-injected eyes (FIGS. 1C-1D).

Previously, it was demonstrated that complement component 3 (C3) is a marker for A1 astrocytes. C3 mRNA and protein levels in ACSA2+ cells isolated from both BSS- and microbead-injected WT and TKO retinas were measured 42 days post-injection. C3 mRNA (FIG. 1E) and protein (FIG. 1F) levels were elevated in WT microbead-injected eyes compared to both BSS-injected eyes and TKO microbead-injected eyes (FIGS. 1E-1F). These data suggest that C3 production in ACSA2+ cells, which encompass both astrocytes and Müller cells, is dependent on IL-1α, TNF-α, and C1q, and that loss of A1 reactivity reduces C3 production.

### Example 2

### TNF-α, IL-1α, and C1q Contribute to Retinal Ganglion Cell Death Secondary to eIOP

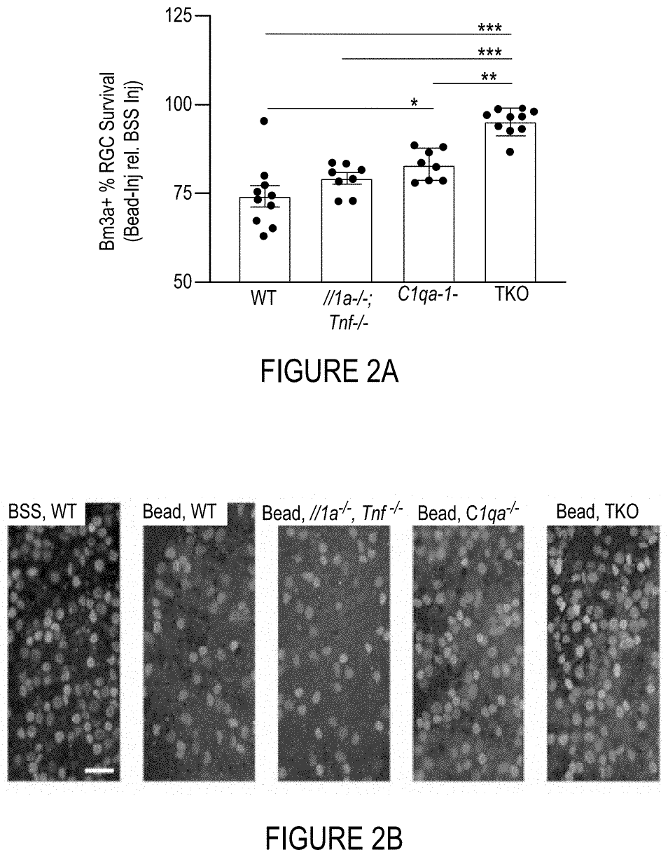

To determine whether TNF-α, IL-1α, and C1q transformation of A1 astrocytes play a role in retinal ganglion cell (RGC) death in the microbead-induced eIOP model of glaucoma, WT. I11a−/−; Tnf-A double knockout (DKO) mice, C1qa−/− single knockout mice, and I11a−/−; Tnf−/−; C1qa−/− (TKO) mice were injected with magnetic microbeads in one eye and BSS in the fellow eye. After 42 days, whole retina flatmounts were stained for the RGC marker Bm3a. Each data point (FIG. 2) represents the RGC count in the microbead-injected eye divided by the RGC count in the BSS-injected eye of the same mouse, multiplied by 100. There was no difference in RGC loss observed in DKO mice compared to WT mice. There was a modest improvement in RGC survival observed in C1qa−/− mice compared to WT mice, consistent with previously published results. RGC death was reduced in TKO mice compared to all other genotypes (FIG. 2), suggesting that the loss of all three cytokines in combination provided an additional benefit beyond the loss of either IL-1α and TNF-α or C1q alone.

### Example 3

### Early Retinal Inflammation is Driven by CD11b+ CD11c+ Cells and Persists Beyond IOP Re-Normalization

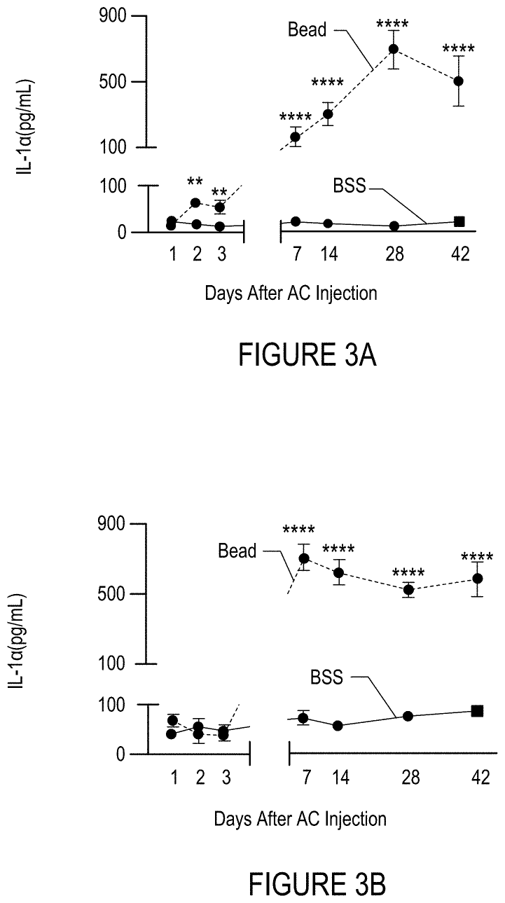

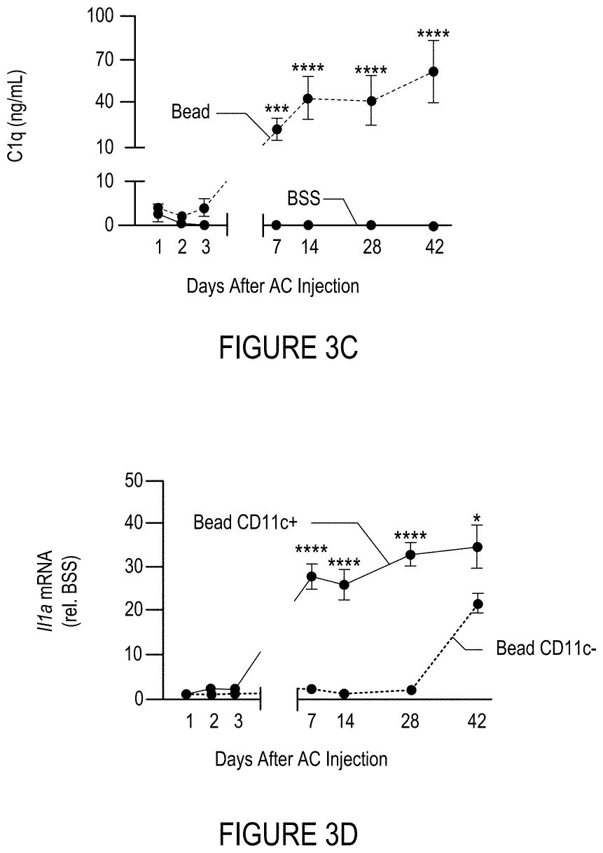

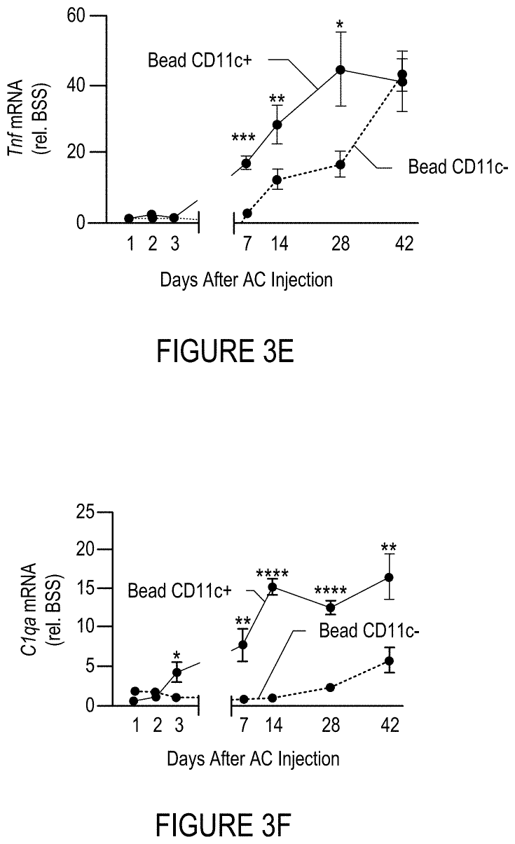

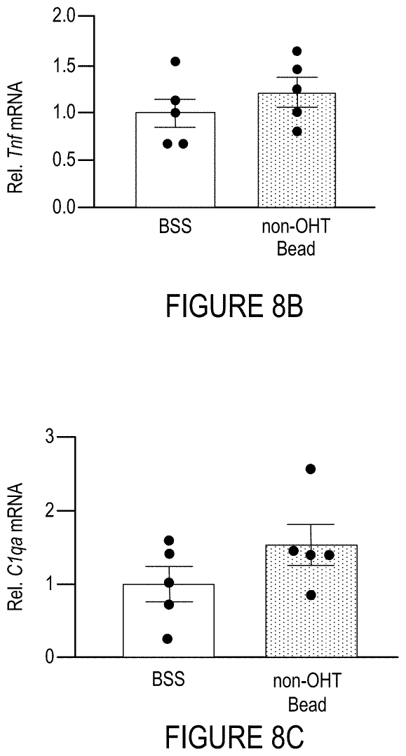

To address the time course and source of IL-1α, TNF-α, and C1q production either microbeads or BSS were injected into the anterior chamber of WT mice. Neurosensory retinas were isolated 1, 2, 3, 7, 14, 28 and 42 days post-injection and were either used to measure IL-1α, TNF-α, and C1q protein levels by ELISA, or dissociated for cell sorting. IL-1α, TNF-α, and C1q protein levels increased in line with IOP, rising by day 7 and then plateauing at subsequent time points (FIGS. 3A-3C). IL-1α, TNF-α, and C1q remained elevated 6 weeks post-injection, despite a return of IOP to baseline levels (FIG. 1A, FIGS. 3A-3C). Microbead-injected eyes that did not have a significant increase in IOP, observed in approximately 5% of eyes, did not have elevated IL-1α, TNF-α, or C1q mRNA levels in CD11b+ cells 42 days post-injection (FIGS. 8A-8C). Together these data suggest that the retinal inflammatory response, initiated by ocular hypertension, can outlast IOP elevation.

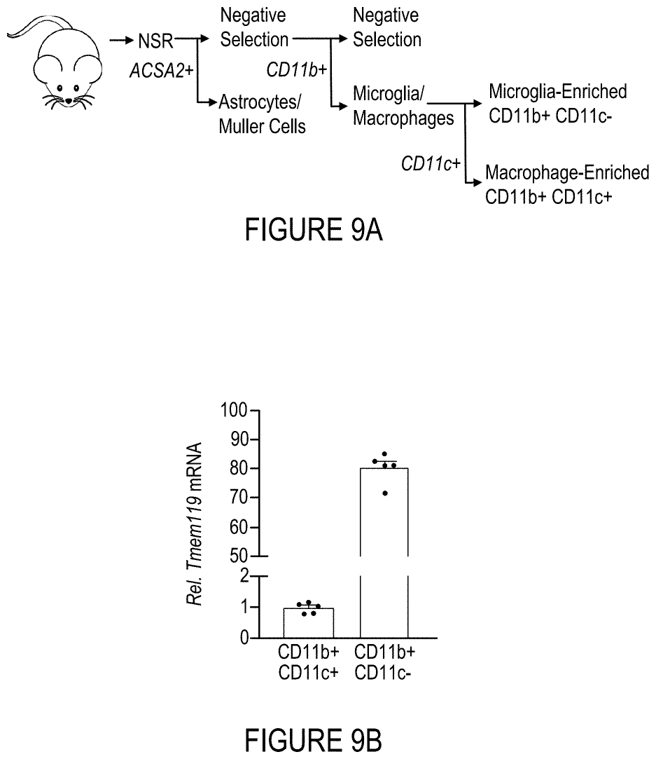

Previous studies have implicated CD11b+ cells in the production of IL-1α, TNF-α and C1q. Recent work in the DBA/2J mouse model of glaucoma has further refined the understanding of CD11b+ cell subpopulations. In the DBA/2J mouse model, early inflammation is driven by CD11b+CD11c+ cells while CD11b+CD11c− cells adopt a largely anti-inflammatory pattern of gene expression. CD11b+ CD11c+ and CD11b+ CD11c− cells (FIG. 9A) were isolated and Tmem19 mRNA levels were measured in both populations (FIG. 9B), a marker of resident microglia. The expression of I11a, Tnf, and C1qa was compared between CD11b+ CD11c− and CD11b+ CD11c+ cells isolated from microbead-injected eyes. Both sets of microbead-injected eyes were normalized against BSS-injected eyes. CD11b+ CD11c+ cells exhibited earlier expression of I11a, Tnf, and C1qa compared to CD11b+ CD11c− cells (FIGS. 3D-3F). Expression of I11a, Tnf, and C1qa by CD11b+CD11c− cells also increased, but lagged behind those of CD11b+ CD11c+ cells by days to weeks (FIGS. 3D-3F). These results suggest that CD11b+ CD11c+ provided the primary source of pro-inflammatory signals in early glaucomatous retinal inflammation following eIOP. In further support of this hypothesis, it was demonstrated that A1 astrocytes were present by 14 days post-injection (FIGS. 1B-1D), when CD11b+ CD11c− cells did not express all three cytokines necessary for A1 transformation (FIGS. 3D-3F). Therefore, initial A1 transformation is unlikely to be driven by CD11b+ CD11c− cells in this model.

### Example 4

### NLY01, a GLP-1R Agonist, Reduces IL-1α, TNF-α, and C1q Production by CD11b+ CD11c+ and CD11b+ CD11c− Cells and Decreases A1 Astrocyte Activation During eIOP

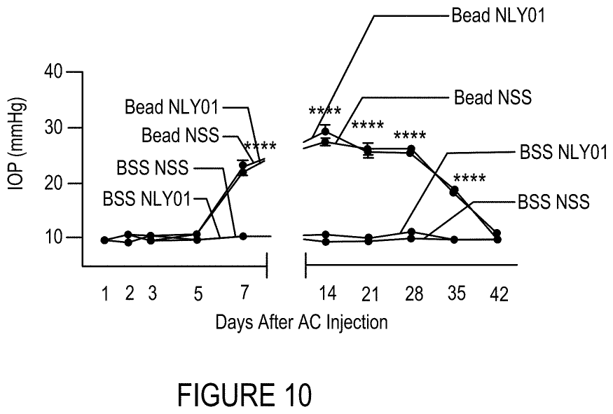

NLY01, a glucagon-like peptide 1 receptor (GLP-1R) agonist, has been shown to modulate microglial phenotype reducing A1 astrocyte activation in the brain in a GLP-1R-dependent manner. It was hypothesized that NLY01 therapy would reduce IL-1α, TNF-α, and C1q production by both microglia and macrophages, thereby decreasing A1 astrocyte conversion secondary to eIOP. To test the efficacy of NLY01 in the mouse model of glaucoma, microbead injections were used to induce eIOP (FIG. 10). Mice were given twice weekly subcutaneous injections of either NLY01 at a dose of 5 mg/kg or normal saline solution (NSS). Neurosensory retinas were harvested 14 and 42 days post-injection to evaluate the effects of NLY01 on both the CD11b+ CD11c+ mediated early response and the CD11b+ CD11c− mediated late response. NLY01 had no effect on intraocular pressure in microbead- or BSS-injected eyes (FIG. 10).

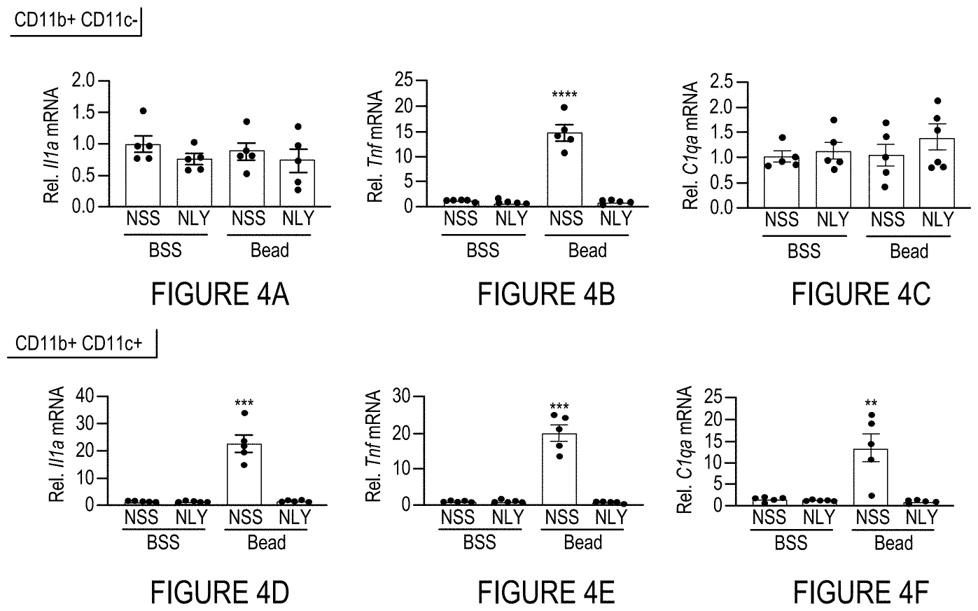

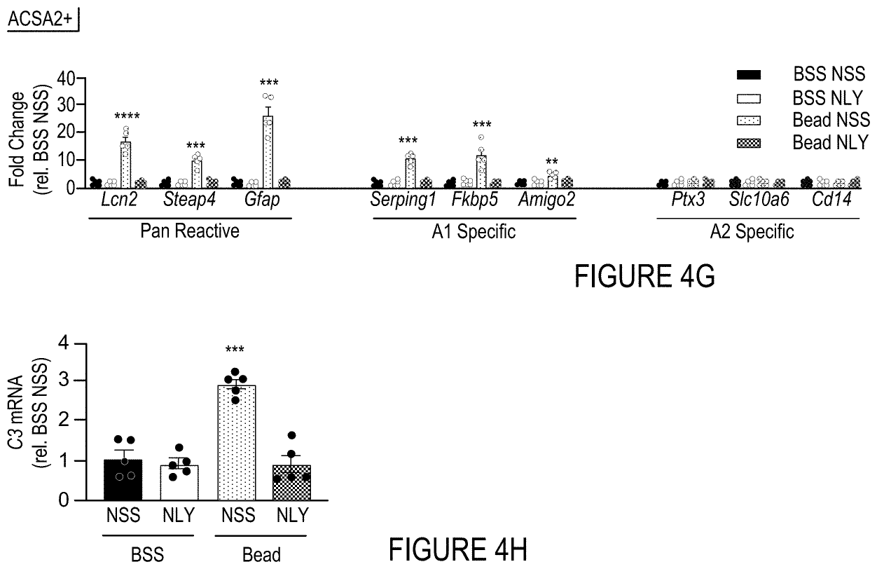

By the 14th day post-injection, NLY01 therapy reduced CD11b+ CD11c− (resident microglia enriched) upregulation of TNF-α (FIG. 4B) without altering basal expressions of IL-1a or C1q (FIGS. 4A, 4C). NLY01 also reduced CD11b+ CD11c+ expression of IL-1α, TNF-α, and C1q (FIGS. 4D-4F). In the ACSA2+(astrocyte and Müller cell enriched) fraction, NLY01 also reduced expression of pan reactive transcripts, A1-specific transcripts, and C3, consistent with decreased A1 activation (FIGS. 4G-4H).

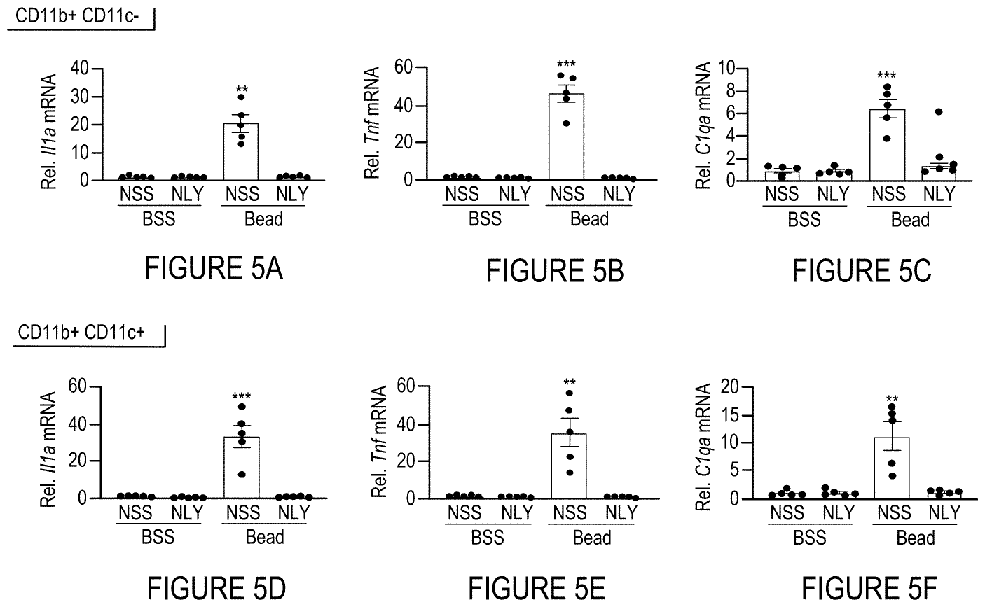

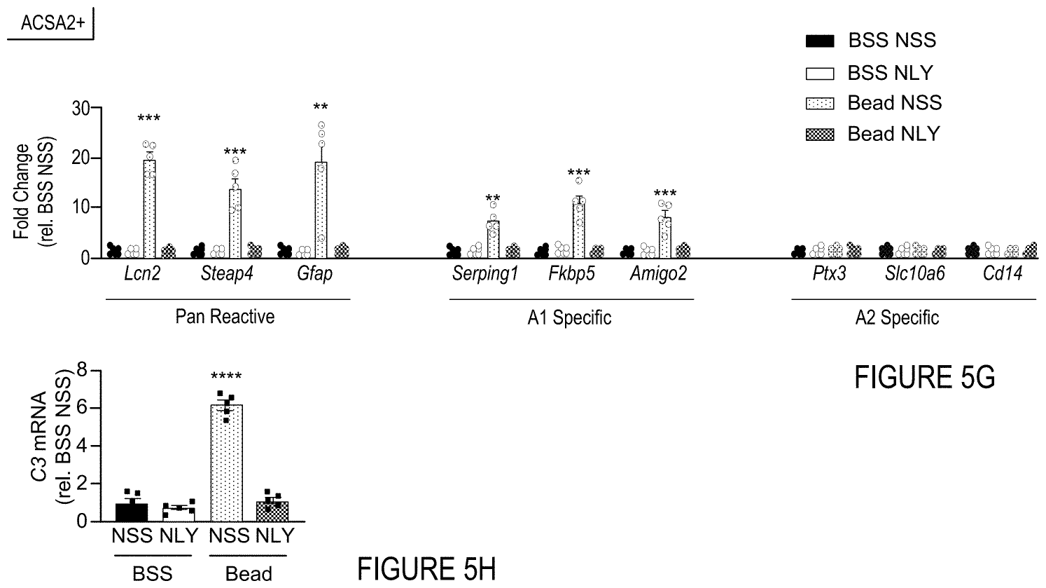

By the 42nd day post-injection, NLY01 therapy reduced both CD11b+ CD11c− (resident microglia enriched) and CD11b+ CD11c− expression of IL-1α, TNF-α, and C1q (FIGS. 5A-5F). NLY01 also reduced expression of pan reactive transcripts, A1-specific transcripts, and C3 in the ACSA2+(astrocyte and Müller cell enriched) fraction (FIGS. 5G-5H), as it had by the 14^{th} day post-injection (FIGS. 4G-41H).

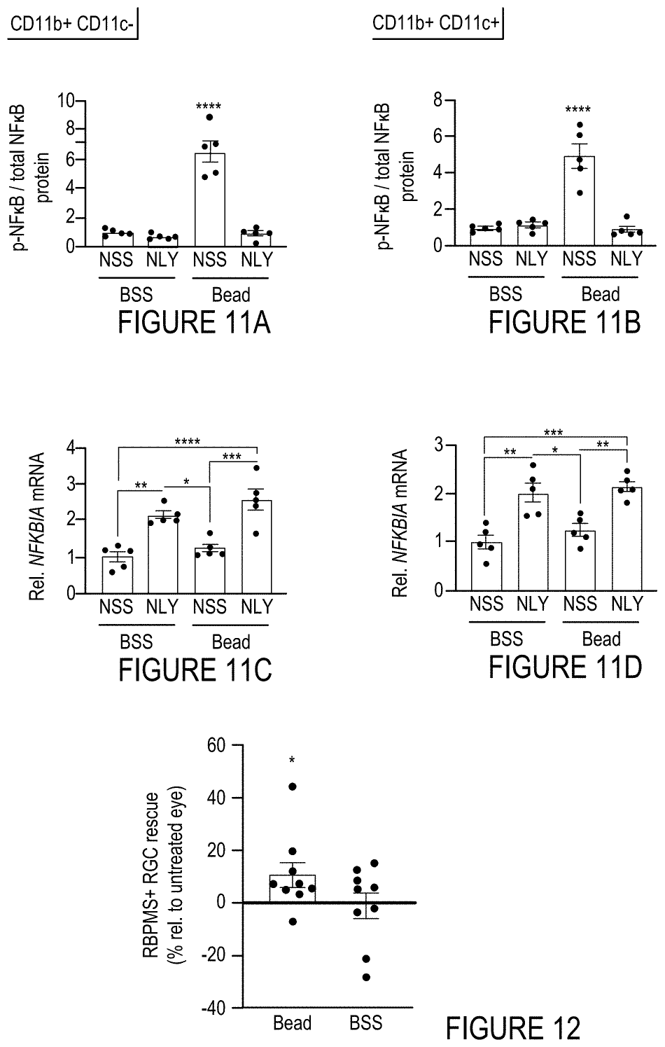

NLY01 has been shown to reduce the nuclear translocation and phosphorylation of the pro-inflammatory transcription factor NFκB in a GLP-1R-dependent manner in brain microglia. At the 42nd day post injection, CD11b+ CD11c− and CD11b+ CD11c+ cells isolated from microbead-injected, NSS treated eyes exhibited an increase in NFκB protein phosphorylation (FIGS. 11A-1I B). NLY01 treatment decreased phosphorylated NFκB protein levels (FIGS. 11A-11B) and increased mRNA levels of IκBα (FIGS. 11C-11D), a negative regulator of NFκB and a known target of GLP-1R. Together these data suggest that NLY01 modulates microglial/macrophage inflammatory phenotype via GLP-1R.

Both eyes of C57BL6/J mice were injected with either microbeads (“Beads”) to increase intraocular pressure (IOP) or with balanced salt solution (“BSS”) to serve as normal pressure controls. One eye of each animal was treated with liraglutide (twice daily to the corneal surface) while the fellow eye was treated with BSS. After 6 weeks, retinal flatmounts were stained for the retinal ganglion cell (RGC) marker RBPMS. These results show that topical treatment with the GLP-1R agonist liraglutide reduced RGC loss in a hypertensive mouse glaucoma model (FIG. 12).

### Example 5

### NLY01 Reduces RGC Death Secondary to eIOP

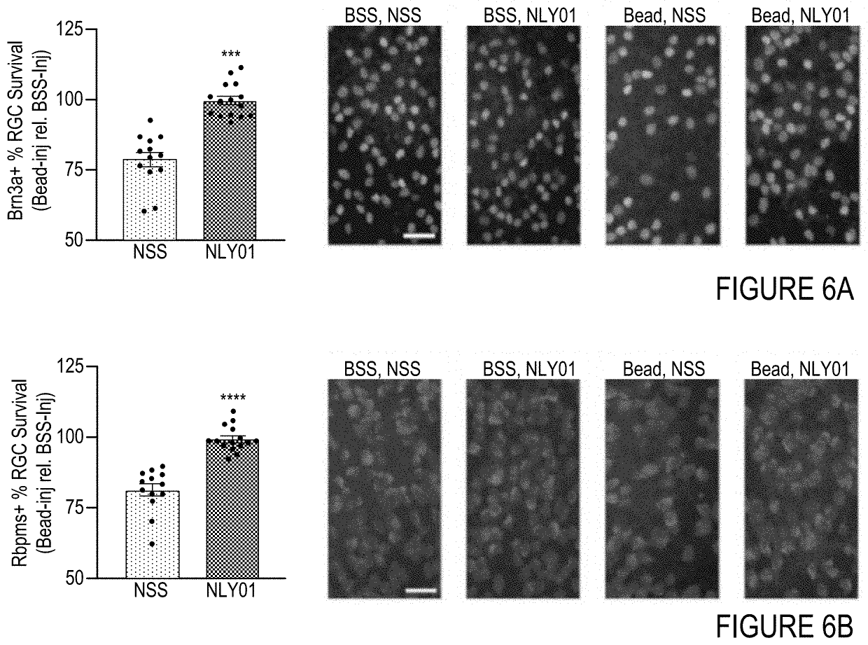

To test the efficacy of NLY01 as a potential neuroprotective agent in the present mouse model of glaucoma, eIOP was once again induced through microbead injections of mice treated with either twice weekly NLY01 at a dose of 5 mg/kg or NSS. After 42 days, neurosensory retinas were isolated, flat-mounted, and labeled with RGC markers Bm3a (FIG. 6A) and Rbpms (FIG. 6B) for RGC counting. Each data point (FIGS. 6A-6B) represents the RGC count in the microbead-injected eye divided by the RGC count in the BSS-injected eye of the same mouse, multiplied by 100. NLY01 therapy reduced RGC death secondary to eIOP, quantified by RBPMS+ and Brn3a+ immunofluorescence and cell counting (FIGS. 6A-6B).

### Discussion

Glaucoma is a disease affecting the retinal ganglion cells and the optic nerve with potentially severe visual implications. Glaucoma is considered more a primary optic neuropathy with secondary effects in the CNS, rather than a primary neurodegeneration affecting the CNS, such as Parkinson's disease, and Alzheimer's disease (AD), amyotrophic lateral sclerosis (ALS) and Huntington's disease (HD). Therapies to slow disease progression are currently limited to IOP reduction through both medical and surgical means. Unfortunately, successful reduction of IOP does not prevent disease progression in a significant number of patients. New therapies targeting other risk factors for glaucoma are needed to prevent irreversible vision loss. The role of A1 reactive astrocytes was examined in the microbead-induced ocular hypertension mouse model of glaucoma. Following induction of ocular hypertension, IL-1α, TNF-α, and C1q production was initially driven by CD11b+CD11c+ cells. The contribution of CD11b+ CD11c− cells to IL-1α, TNF-α, and C1q production was not observed until weeks to months after ocular injection. Together, these three cytokines triggered the formation of A1 astrocytes as demonstrated by upregulation of A1-specific transcripts and C3 production in an ACSA2+ retinal cell population (enriched for astrocytes and Müller cells). Treatment with the GLP-1R agonist, NLY01, reduced microglia/macrophage production of IL-1α, TNF-α, and C1q, decreased A1 astrocyte conversion, and protected against RGC death in this mouse model of glaucoma.

Recent work corroborates several critical findings described herein. Using both optic nerve crush and microbead injections, it was demonstrated that I11a−/−; Tnf−/−; C1qa−/− (TKO) mice exhibit significant reductions in RGC death at rates comparable to the findings described herein. Further, preserved RGCs are functionally intact under examination by in vivo electrophysiology. In combination, these results highlight the neurotoxic role of IL-1α, TNF-α, and C1q in RGC death following injury. The finding that rescued RGCs remain functionally viable lends further credence to inhibition of A1 astrocyte transformation as a possible therapy for glaucoma.

Transcriptomic data from the DBA/2J mouse model of glaucoma suggest that early inflammation is driven by CD11b+ CD11c+ cells, while CD11b+ CD11c− cells initially adopt an anti-inflammatory pattern of gene expression. The results described herein support this finding by demonstrating that that CD11b+ CD1l c+ cells upregulated IL-1α, TNF-α, and C1q expression prior to contribution from CD11b+ CD11c− cells. Results suggest that CD11b+ CD11c+ cells are early contributors to A1 astrocyte formation following elevated IOP (eIOP). Transcriptomic data from FACS isolated CD11b+ CD11c+ retinal cells from the DBA/2J mouse model of glaucoma demonstrate that this population is enriched for infiltrating macrophages compared to other blood-borne immune cells and resident microglia. It should be noted that the embryonic origin of the CD11b+ CD11c+ cell population has not been conclusively demonstrated, and these cells could therefore represent resident retinal microglia that have undergone a state change, infiltrating macrophages, or a mixture of both. However, macrophage infiltration has been implicated in the pathogenesis of glaucoma. Specifically, macrophages have been observed in sections of human glaucomatous retina and optic nerve in both mild and severe cases. Progression of visual field loss in normotensive glaucoma also is associated with increased systemic levels of macrophage chemoattractant protein-1 (MCP-1), a potent chemotactic factor for monocytes. Although the presence of the blood-retina barrier confers a degree of immune-privilege to the retina, disruption of the blood-retina barrier has been observed in diseases of ocular inflammation. Glaucomatous retinas often exhibit focal bleeds in the nerve fiber layer surrounding the optic nerve head. These so-called Drance hemorrhages disrupt the blood-retina barrier, and present an opportunity for blood-borne immune cells, such as macrophages, to enter the retina. Together these data provide an impetus for work characterizing the origin of CD11b+ CD11c+ cells in mouse models of glaucoma, as well as the role of infiltrating macrophages and the integrity of the blood-retina barrier in glaucoma.

Following microbead injections, the time course of IL-1α, TNF-α, and C1q upregulation corresponds to the trajectory of IOP increase, lending support to eIOP as the initiator of inflammation. Despite a return to normal IOP, pro-inflammatory cytokines remained upregulated at 6 weeks post-injection, suggesting that the inflammatory pathway remains active beyond the inciting eIOP. In human glaucomatous eyes, a reduction in IOP, whether by pharmacological or surgical means, is not always sufficient to prevent further RGC degeneration. The data herein suggest that persistent inflammation after normalization of IOP may contribute to these refractory cases, presenting treatment opportunity for patients who have exhausted therapies rooted in IOP reduction.

A1 astrocytes upregulate complement component 3 (C3), and C3 inhibitors were shown to reduce RGC cell death in the DBA/2J mouse model of glaucoma. Elevated C3 production following A1 activation and decreased C3 production following NLY01 inhibition was demonstrated in the present glaucoma model. Importantly, C3 is not the only source of toxicity from A1 astrocytes. While C3 inhibition offers some RGC protection, prevention of A1 astrocyte formation may confer more complete protection against eIOP.

Within two weeks of microbead injection, pro-inflammatory CD11b+ CD11c+ cells upregulate IL-1α, TNF-α and C1q expression, which triggers A1 astrocyte transformation. NLY01 blocks this pathway and rescues RGCs from eIOP-induced death at 6 weeks post-injection. Previous work demonstrates that RGC loss does not occur in the microbead model until after 4-6 weeks of prolonged eIOP. This delay between A1 transformation and RGC death raises the question of whether RGC-autonomous mechanisms of cell stress must act in concert with the non-cell autonomous mechanism of retinal inflammation to trigger RGC death. Loss of one of these two pathways, conferred by NLY01 administration in the present study, was sufficient to rescue RGCs. Several observations support this multi-hit hypothesis. First, in animal models of unilateral glaucoma, where one eye has eIOP and the other eye with normal IOP is used as an internal control, microglial activation and inflammation can be observed without RGC loss in the control optic nerve. Second, neuronal injury, either via optic nerve crush or eIOP, is a necessary precursor for astrocyte-mediated neuroinflammatory cell death. Targeting both RGC-autonomous mechanisms of stress and retinal inflammation may act in a synergistic fashion to rescue additional RGCs.

Elevated intraocular pressure induces deficits in axon transport along the optic nerve and reduction in NaV1.2 protein levels in RGCs after 4 weeks of ocular hypertension. In contrast, changes in RGC electrical signaling occur earlier after eIOP. Following just two weeks of eIOP, RGCs exhibit increased electrical responses to preferred stimuli in both light onset and offset cells. During this same two-week period, RGCs exhibit excessive dendritic pruning. In both the brain and the retina, dendritic pruning is linked to the production of C1q, and the subsequent initiation of the classical complement cascade by CD11b+ cells. The results herein show that CD11b+ CD11c+ cells, and not CD11b+ CD11c− cells, are responsible for C1q upregulation and possibly downstream dendritic pruning. NLY01 reduced C1qa expression in CD11b+ CD11c+ cells 2 weeks after IOP elevation, suggesting that NLY01 may also exert effects on RGCs by preventing synaptic pruning in RGCs. A1 astrocytes themselves may also directly promote aberrant electrical signaling, as in vitro work has shown that A1 astrocytes reduced the number of synapses, mEPSC frequency, and mEPSC amplitude in cultured RGCs.

In the present study, microbeads were injected into the anterior chamber of one mouse eye while the contralateral eye was injected with BSS. This paired approach reduced the impact of biological variability through the use of an internal control. However, recent work has shown that unilateral induction of eIOP in rats resulted in microglial reactivity throughout the visual pathway, including in the contralateral, normotensive optic nerve. While most of the present experiments normalized the microbead eye against the normotensive control eye, one exception to this can be found in FIGS. 3A-3C where absolute protein levels were measured using ELISA. Here, normotensive eyes showed no upregulation of IL-1α, TNF-α, and C1q proteins, suggesting that if present, microglial reactivity in the control eye may not result in A1 astrocyte activation in this model of glaucoma. NLY01 reduces microglial/macrophage activation and prevents A1 astrocyte formation. NLY01 belongs to a family of glucagon-like peptide 1 receptor (GLP-1R) agonists. NLY01 is a long-acting GLP-1R agonist that efficiently penetrates the blood-brain barrier. In mouse models of Parkinson's disease, NLY01 concentrations in the brain are higher than in WT animals. This increase in NLY01 concentration was attributed to blood-brain barrier breakdown present in the Parkinson's disease mouse models. Similarly, blood-retina barrier disruptions in glaucoma may serve a therapeutic benefit by providing a gateway for systemically administered therapeutics to access the retina and the optic nerve. This further highlights the need to characterize the state of the blood-retina barrier in glaucoma.

Previous work has shown that NLY01's effects on CD11b+ cells are not limited to reductions in levels of IL-1α, TNF-α, and C1q. Rather, NLY01 treatment reduces microglia density and relative IBA1 levels in a mouse model of Parkinson's disease, suggesting a broader immunosuppressive effect. It is therefore possible that the RGC rescue observed following NLY01 treatment can be partially attributed to a reduction in microglial/macrophage reactivity, and occurs independent of the drug's effect on astrocyte phenotype. This is consistent with prior findings demonstrating that reduced CD11b+ cell reactivity protects RGCs from the effects of eIOP.

The tolerability and efficacy of NLY01 in humans is currently being tested in a clinical trial for Parkinson's disease. NLY01 belongs to the GLP-1R class of therapeutic agonists that have been used in the clinic for over 15 years. During that time, GLP-1R agonists have demonstrated a favorable safety profile in the long-term treatment of type 2 diabetes mellitus. Diabetes is a known risk factor in glaucoma, and GLP-1R agonists' wide usage in diabetes treatment presents an opportunity to retrospectively evaluate its effects among patients with coexisting glaucoma in a large-scale observational study. The findings of such a study could provide evidence as to whether existing GLP-1R agonists affect glaucoma incidence or progression among patients with diabetes.

Glaucoma is a group of diseases with disparate but often interlinking etiologies. The present data highlight neuroinflammation as a mechanism of glaucomatous damage and demonstrate rescue by NLY01 through the drug's ability to decrease retinal inflammation. Current therapies targeting IOP are not sufficient to prevent vision loss in many glaucoma patients. NLY01, or more broadly the class of GLP-1R agonists, may be a fruitful avenue for future exploration.

### Limitations

RGC degeneration occurs in retrograde fashion, beginning with the retraction of RGC synaptic terminals in the colliculus, followed by axonal degeneration, with loss of RGC soma constituting a late step in the degenerative cascade. The present study does not include optic nerve analysis, and it is important to consider alternative explanations for these findings given this limitation. Rbpms, and to a lesser degree, Brn3a immunolabeling captures most but not all RGC somas, and it is possible that rescue is more robust than indicated by soma analysis alone. Conversely, it is possible that RGC counts underestimated RGC death by counting RGC soma that have yet to degenerate. It is also possible that ablation of A1 astrocytes prolonged expression of RGC markers in the soma leading to overcounting of RGCs in TKO and NLY01 treated animals. It should be noted that the rescued RGCs in TKO animals have been shown to be electrically and functionally intact. NLY01 treatment decreases IL-1α, TNF-α, and C1q expression in a similar fashion to TKO, lending credence to the possibility that it rescues RGCs and preserves their functionality in a similar fashion. Nevertheless, it cannot be said definitively that this is the case following NLY01 treatment until the optic nerve has been evaluated, which is planned to be pursued in future experiments.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

1. A method for reducing retinal inflammation and neuron death secondary to ocular hypertension in a subject in need thereof, the method comprising administering to the subject a therapeutically effective amount of a glucagon-like peptide-1 receptor (GLP-1R) agonist.

2. The method of claim 1, wherein the GLP-1R agonist is selected from the group consisting of exenatide, a pegylated exendin 4, NLY01, lixisenatide, liraglutide, albiglutide, dulaglutide, and semaglutide.

3. (canceled)

4. The method of claim 1, further comprising reducing the ocular hypertension from an intraocular pressure (IOP) of greater than 21 mm Hg by 20% to 30%.

5. The method of claim 4, wherein the intraocular pressure is reduced by topically administering an ocular hypotensive medication to an eye of the subject.

6. The method of claim 5, wherein the ocular hypotensive medication is a prostaglandin analog, a beta-adrenergic blocking agent, an alpha adrenergic agonist, a carbonic anhydrase inhibitor, a Rho kinase inhibitor or combinations thereof.

7.-12. (canceled)

13. The method of claim 4, wherein the intraocular pressure (IOP) is reduced by performing glaucoma surgery on an eye of the subject.

14. (canceled)

15. The method of claim 5, wherein the GLP-1R agonist is administered to the subject following administration of the ocular hypotensive medication to the eye of the subject, wherein the ocular hypertension is reduced to a normal intraocular pressure of less than 21 mm Hg.

16. The method of claim 13, wherein the GLP-1R agonist is administered to the subject following the glaucoma surgery on the eye of the subject, wherein the ocular hypertension is reduced to a normal intraocular pressure of less than 21 mm Hg.

17. A method for reducing production of IL-1α, TNF-α, and C1q by CD11b+ CD11c+ and CD11b+ CD11c− cells during elevated intraocular pressure (IOP) in a subject, the method comprising administering to the subject a therapeutically effective amount of a glucagon-like peptide-1 receptor (GLP-1R) agonist.

18. The method of claim 17, wherein the GLP-1R agonist is selected from the group consisting of exenatide, a pegylated exendin 4, NLY01, lixisenatide, liraglutide, albiglutide, dulaglutide, and semaglutide.

19. (canceled)

20. The method of claim 17, further comprising reducing the ocular hypertension from an intraocular pressure (IOP) of greater than 21 mm Hg by 20% to 30%.

21. The method of claim 20, wherein the intraocular pressure is reduced by topically administering an ocular hypotensive medication to an eye of the subject.

22. The method of claim 21, wherein the ocular hypotensive medication is a prostaglandin analog, a beta-adrenergic blocking agent, an alpha adrenergic agonist, a carbonic anhydrase inhibitor, a Rho kinase inhibitor or combinations thereof.

23.-28. (canceled)

29. The method of claim 20, wherein the elevated intraocular pressure (IOP) is reduced by performing glaucoma surgery on an eye of the subject.

30.-32. (canceled)

33. A method for decreasing transformation of astrocytes to an A1 neurotoxic phenotype (A1 astrocytes) and activation of the A1 astrocytes in a retina of a subject and decreasing production of complement component 3 (C3) by A1 astrocytes during elevated intraocular pressure (eIOP), the method comprising administering to the subject a therapeutically effective amount of a glucagon-like peptide-1 receptor (GLP-1R) agonist.

34. The method of claim 33, wherein the GLP-1R agonist is selected from the group consisting of exenatide, a pegylated exendin 4, NLY01, lixisenatide, liraglutide, albiglutide, dulaglutide, and semaglutide.

35. (canceled)

36. The method of claim 33, further comprising reducing the ocular hypertension from an intraocular pressure (IOP) of greater than 21 mm Hg by 20% to 30%.

37. The method of claim 36, wherein the intraocular pressure is reduced by topically administering an ocular hypotensive medication to an eye of the subject.

38. The method of claim 37, wherein the ocular hypotensive medication is a prostaglandin analog, a beta-adrenergic blocking agent, an alpha adrenergic agonist, a carbonic anhydrase inhibitor, a Rho kinase inhibitor or combinations thereof.

39.-44. (canceled)

45. The method of claim 36, wherein the intraocular pressure (IOP) is reduced by performing glaucoma surgery on an eye of the subject.

46.-48. (canceled)

49. A method for reducing retinal ganglion cell (RGC) death secondary to elevated intraocular pressure (eIOP), decreasing or eliminating overexpression of IL-1α, TNF-α, and C1q in a retina, and decreasing A1 astrocyte transformation in the retina of a subject having eIOP, the method comprising administering to the subject a therapeutically effective amount of an adenoviral vector comprising a triple knockout of genes encoding IL-1α, TNF-α, and C1q (I11a−/−; Tnf−/−; C1qa−/−).

50. The method of claim 49, wherein the adenoviral vector comprising the I11a−/−; Tnf−/−; C1qa−/− is constructed by CRISPR-cas9 based genome editing to delete I11a; Tnf; and C1qa.
